(19) United States
(12) Patent Application Publication
(10) Pub. No.: US 2012/0179249 A1
(43) Pub. Date: Jul. 12, 2012

(54) ACCOMMODATING INTRAOCULAR LENS AND METHODS OF USE

(75) Inventor: D. Jackson Coleman, Haworth, NJ (US)

(73) Assignee: CORNELL UNIVERSITY, Ithaca, NY (US)

(21) Appl. No.: 13/363,863

(22) Filed: Feb. 1, 2012

Related U.S. Application Data
(63) Continuation-in-part of application No. 11/572,061, filed on May 5, 2008.

(51) Int. Cl. A61F 2/16

(52) U.S. Cl. 623/6.43

(57) ABSTRACT

The present invention relates to an accommodating intraocular lens, which is suitable for replacement of the natural crystalline lens of the eye within the capsular bag. The intraocular lens can include a retainer plate with an annular region surrounding a central opening, and an optical lens. The retainer plate acts as a foundation for the optical lens, as the retainer plate can be positioned near or at the posterior portion of the lens capsule. The optical lens can sit on the retainer plate, but is otherwise not attached to the retainer plate. The optical lens can overlap or fit within the central opening of the retainer plate. The retainer plate can include a lip portion integrally disposed on a region of the retainer plate, wherein the lip provides a surface for holding at least a portion of an intraocular lens.

This application is a continuation-in-part of co-pending U.S. Non-Provisional patent application Ser. No. 11/572,061, filed May 5, 2008 as a national application based on PCT Application No. PCT US05/25748, filed Jul. 25, 2005, which claims the priority benefit of U.S. Provisional Patent Application Ser. No. 60/590,078, filed Jul. 22, 2004, which are hereby incorporated by reference in their respective entireties.

### 1. FIELD OF INVENTION

Embodiments of the invention relate to intraocular lenses (IOLs) including accommodating IOLs, structures for enhancing the operation of an IOL including accommodating IOLs, methods of implanting intraocular lenses and/or enhancer apparatus into an in-vivo, aphakic eye, and methods of replacing intraocular lenses that have been implanted into a patient's eye.

### 2. BACKGROUND OF INVENTION

Cataracts are currently the leading cause of blindness among adults in the United States. A cataract is a clouding of a part of the eye known as the crystalline lens. Although cataracts result from many conditions, the most frequent cause is the natural aging process. Most people develop some clouding of the lens after the age of 60. About 50 percent of Americans ages 65 to 74 have cataract, and about 70 percent of those age 75 and over have it. As there are growing numbers of elderly in the United States, the incidence of cataracts is increasing. Other causes of cataracts may include injury, chronic eye disease, and other system-wide diseases such as diabetes.

Surgery to remove the diseased crystalline lens is the only effective treatment for cataract. Neither diet nor medications have been shown to stop cataract formation. Cataract surgery is now a frequently performed operation in most parts of the world. More than 1.5 million cataract procedures are performed every year in the U.S. alone, and, in the majority of those cases, the diseased tissue is replaced with an artificial device known as an intraocular lens implant. This surgery is very successful in restoring vision—more than 95 percent of people who have a cataract removed have no complications and end up with better vision. Many people report not only better vision but also a reduction in the power of their lens prescription and improvements in their overall quality of life after the operation.

Presbyopia, a condition in which the natural lens of the eye becomes thicker and less flexible, is also a symptom of the natural aging process. Patients with presbyopia lose the ability of ocular focus due to lack of accommodation ability of the natural lens. Replacement of the natural crystalline lens with an artificial intraocular lens is one emerging treatment for patients with presbyopia, although generally only with accompanying cataract.

Intraocular lenses for cataract lens replacement surgery differ widely in their physical appearance and arrangement. Intraocular lenses have been described. in U.S. Pat. Nos. 4,254,509, 4,298,996, 4,409,691, 4,424,597, 4,573,998, 4,664,666, 4,673,406, 4,738,680, 4,753,655, 4,778,463, 4,813,955, 4,840,627, 4,842,601, 4,963,148, 4,994,082, 5,047,051, 5,674,282, 6,342,073, 6,387,126, 6,451,056, and U.S. Patent Application Publication No. 2003/0050696.

Two general classes of intraocular lenses—accommodating and non-accommodating—have been developed. Non-accommodating intraocular lenses cannot change shape or position in the eye and, therefore, do not operate in conjunction with the natural accommodation capabilities of the human eye, whereas accommodating lenses do have at least some capability to operate in conjunction with the natural accommodation capabilities of the eye or by built-in multifocal properties. Accommodation involves alternating relaxation and constriction of the ciliary muscle by the brain to provide the eye with near and distant vision. This ciliary muscle action is automatic and shapes the natural crystalline lens to the appropriate optical configuration for focusing on the retina the light rays entering the eye from the scene being viewed. Ocular mechanics of natural eye accommodation are also governed by zonular ligaments connecting the ciliary muscle to the perimeter of the lens and vitreous material that fills the posterior segment of the eye.

Most non-accommodating intraocular lenses have single focus optics that focus the eye at a certain fixed distance only. Other non-accommodating lenses have bifocal or multifocal optics, which image both near and distant objects on the retina of the eye. The brain selects the appropriate image and suppresses the other image, so that a bifocal intraocular lens provides both near vision and distant vision without eyeglasses. Bi- or multifocal intraocular lenses, however, suffer from the disadvantage that each image represents only about 40% of the available light, and as much as 20% of the light is lost in scatter.

A common design feature of the available accommodative intraocular lenses is to translate the optic forward in the eye. For example, the accommodating intraocular lens disclosed by U.S. Pat. No. 5,674,282 to Cumming is made of a lens body having a central optical zone or optic and plate haptics extending from diametrically opposite edges of the optic. Ciliary muscle contraction exerts opposing endwise compression forces on the ends of the lens haptics with resultant endwise compression of the lens. Relaxation of the ciliarly muscle causes retraction of the lens. Accommodating intraocular lenses designed to translate the optic forward in the eye fail to address an important feature of natural lens accommodation. That is, natural accommodation benefits considerably from changes in surface curvatures. Available accommodating intraocular lenses fail to possess the capability of undergoing changes in surface curvature. In other words, available accommodating intraocular lenses do not uniformly flex and move or maintain their flexibility with in-growth of fibroblasts around the supporting capsular structure of the eye. Furthermore, available accommodating intraocular lenses are not amenable to removal or replacement of the optical lens because of adhesion of the lens to the capsular/zonular support structure of the eye.

The embodiments of the present invention are directed to overcoming these and other limitations in the art.

### SUMMARY OF THE INVENTION

One aspect of the present invention relates to an intraocular lens having a retainer plate with an annular region surrounding a central opening and an optical lens removably attached to the retainer plate within the central opening.

Another aspect of the present invention relates to a method of implanting an intraocular lens into a patient's eye. This method involves providing an intraocular lens having a retainer plate with an annular region surrounding a central opening and an optical lens removably attached to the retainer plate within the central opening. The intraocular lens is implanted into the patient's eye.

A further aspect of the present invention relates to a method of replacing an intraocular lens that has been implanted into a patient's eye. This method involves removing the intraocular lens from the patient's eye. The intraocular lens has a retainer plate with an annular region surrounding a central opening and a first optical lens removably attached to the retainer plate within the central opening. The first optical lens is removed from the retainer plate. A second optical lens is attached to the retainer plate within the central opening. The intraocular lens with the second optical lens is implanted into the patient's eye.

Yet another aspect of the present invention relates to an intraocular lens having a retainer plate with an annular region surrounding a central opening, where the annular region is porous or perforated, and an optical lens integral with the retainer plate within the central opening.

Still another aspect of the present invention relates to a method of implanting an intraocular lens into a patient's eye. This method involves providing an intraocular lens having a retainer plate with an annular region surrounding a central opening, where the annular region is porous or perforated, and an optical lens integral with the retainer plate within the central opening. The intraocular lens is implanted into the patient's eye.

An embodiment of the invention is an enhancer for an in-vivo intra-ocular lens (IOL). According to an exemplary aspect, the enhancer includes a retainer plate having an annular region comprising an anterior surface and a posterior surface surrounding a central clear aperture, wherein the retainer plate has a groove in at least a portion of the inner perimetal surface of the annular region into which a corresponding peripheral region of an IOL can engage.

A related embodiment is an enhancer for an in-vivo intra-ocular lens (IOL), comprising a retainer plate having an annular region comprising an anterior surface and a posterior surface surrounding a central clear aperture; a lip projecting from a region of the anterior surface of the retainer plate, wherein the lip provides a contact surface for at least a portion of an IOL border or rim, or haptic. In exemplary, non-limiting aspects:
the lip is located adjacent an outer perimeter region of the retainer plate;
the lip is continuous and centered about the clear aperture;
the lip is segmented;
   the lip comprises at least two segments disposed in opposing relationship and centered about the clear aperture;
   at least two opposing lip segments are located at least about 1 millimeter (mm) from the inner perimeter of the retainer plate;
the lip is located adjacent an inner perimeter of the retainer plate;
   the lip is located at least about 1 millimeter (mm) from the inner perimeter of the retainer plate;
the lip is located intermediate the outer perimeter and the inner perimeter of the retainer plate;
the enhancer further comprises a lip projecting from a region of the posterior surface of the retainer plate;
   the enhancer further comprises at least one lip segment;
the retainer plate is characterized by a flexibility sufficient to allow its insertion into a capsular bag;
the lip has a thickness that is greater than the thickness of the retainer plate;
the lip has a thickness that is about 0.5× to about 5× of the retainer plate's thickness;
   the retainer has a thickness between about 50 to 100 micrometers (μm) and the lip has a thickness between about 100 to 500 μm;
the lip has a flat inner surface;
the lip has a hooked shape;
the retainer plate is perforated;
the retainer plate has a diameter of between about 10 mm to about 15 mm;
the annular region has a diameter of between about 4 mm to about 6 mm;
the retainer plate is medicated;
   the retainer plate is medicated with at least one of an antifibroplastic agent, cyclosporine, and mitomycin.

An embodiment of the invention is directed to a method that involves the steps of inserting an enhancer as described hereinabove into a capsular bag of an in-vivo, aphakic eye, and positioning an IOL adjacent the anterior surface of the retainer plate in a manner such that the IOL is held in operable contact by at least a portion of the lip of the retainer plate. In various exemplary, non-limiting aspects:
the IOL is an accommodating IOL;
the method involves positioning the IOL after inserting the enhancer.

Embodiments of the invention provide an improved accommodating intraocular lens assembly or components thereof, for use in an aphakic eye. In particular, the retainer plate provides natural accommodation benefits. By having a separate retainer plate and optical lens, the inventory of optimal plate size and optic power allows proper sizing for both globe size and dioptic lens power. The diameter of the retainer plate is determined by measurement of the diameter of the natural or crystalline lens before extraction. Lens power is determined separately and independently although the expected lens position post surgery can be derived from the measurement of natural lens dimension. This can improve calculation of lens power. The plate holding the intraocular lens of the present invention also is designed to retard fibrous in-growth, thereby keeping a clear central area for optimal acuity.

Embodiments of the invention utilize the entire vitreo-zonular diaphragm in order to support and enhance accommodating intraocular lens movement by taking advantage of the uniform pressure exerted by the vitreous body on the lens capsule, and the enhancer or retainer plate/optical lens within the capsule. The retainer plate can augment vitreous support and forward movement of the intraocular lens with accommodation. Embodiments of the invention thus overcome the problems of conventional intraocular lens constructions that utilize only a portion of the vitreous support allowing a bulging to the unsupported sides of the haptics that are used in these conventional intraocular lens constructions, thus dissipating the vitreous support. Ridges may be incorporated into the retainer plate for the purpose of inhibiting epithelial ingrowth. The central opening of the retainer plate is also useful in preventing ingrowth, allowing unimpeded vision and, when necessary, allowing laser opening of a cloudy posterior capsule. The diameter of the retainer plate would be measured preoperatively based on the lens size, which is determined using ultrasound or optical techniques, and should ‘fit’ the posterior capsule and the horizontal dimension of the ciliary diameter as so measured.

### BRIEF DESCRIPTION OF THE DRAWINGS

FIG. 1 is a plan view of a first embodiment of the intraocular lens of the present invention;

FIG. 2 is a cross-sectional side view of one embodiment of the intraocular lens of FIG. 1, in which the optical lens is removably attached to the retainer plate by providing the optical lens with an annular groove into which the retainer plate fits;

FIG. 3 is a cross-sectional side view of a second embodiment of the intraocular lens of FIG. 1, in which the optical lens is removably attached to the retainer plate by providing the retainer plate with an annular groove into which the optical lens fits;

FIG. 4A is an exploded, perspective view of the intraocular lens of FIG. 1, in which the optical lens has flanges on its anterior surface to prevent posterior displacement when the optical lens is removably attached to the retainer plate. The optical lens also has flanges on its posterior surface to prevent anterior displacement when the optical lens is removably attached to the retainer plate. FIG. 4B is a cross-sectional side view of the intraocular lens of FIG. 4A;

FIG. 5 is a cross-section of a human eye, showing the positioning and attachment of the natural crystalline lens;

FIG. 6 is a cross-section of a human eye in which the natural crystalline lens has been removed and an intraocular lens of the present invention has been surgically implanted;

FIG. 7A is a plan view of a second embodiment of the intraocular lens of the present invention in which the porous retainer plate and the optical lens are integral. FIG. 7B is a cross-sectional view of the intraocular lens of FIG. 7A; and

FIG. 8a is a cross-sectional side view of an enhancer; FIG. 8b shows an alternative form of the lip of the enhancer; FIG. 8c shows another alternative form of the lip of the enhancer, according to exemplary aspects of the invention; and

FIG. 9 is a front elevation view of the enhancer of FIG. 8a showing an IOL positioned in the enhancer, according to an illustrative aspect of the invention.

### DETAILED DESCRIPTION OF EXEMPLARY EMBODIMENTS OF THE INVENTION

One aspect of the present invention relates to an intraocular lens having a retainer plate with an annular region surrounding a central opening and an optical lens removably attached to the retainer plate within the central opening.

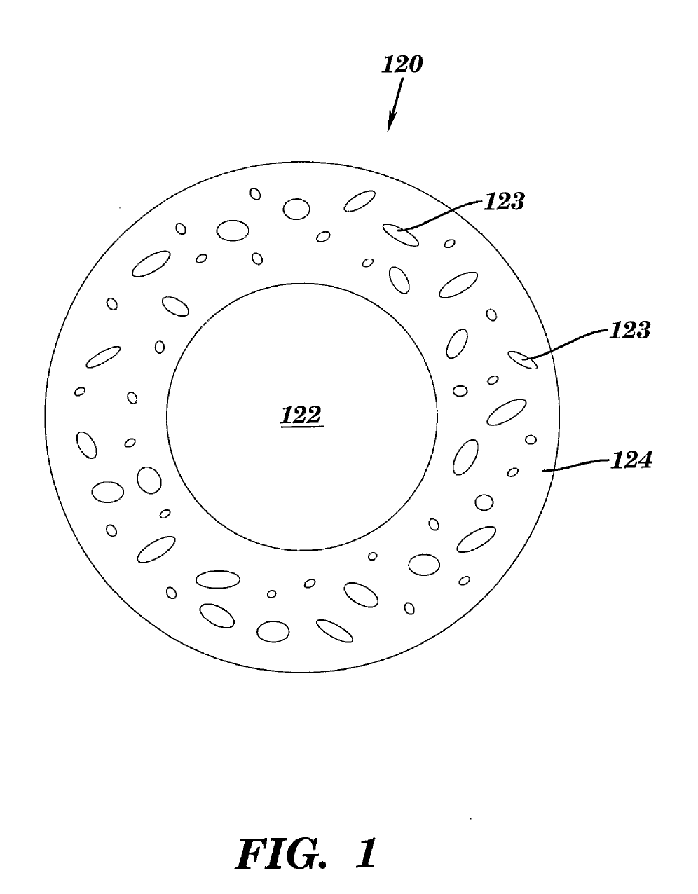

FIG. 1 is a plan view of the intraocular lens of the present invention. Intraocular lens 120 has optical lens 122, which is removably attached to retainer plate 124. Either or both of optical lens 122 and retainer plate 124 are constructed of a flexible material including, without limitation, acrylic, silicone, or poly(methylmethacrylate) (“PMMA”), or mixtures thereof. Flexible material allows accommodation of the intraocular lens by flexing with ciliary muscle contraction and vitreous pressure. As a result, intraocular lens 120 has the ability to adjust to surface curvatures. The optical power of a lens or a surface is determined to some extent by the curvature of the surface. An increase or steepening of the curvature (a decrease in the radius of curvature) will result in an increase in the optical power. An optic which can flex to increase the curvature of one or both surfaces (decrease the radius of curvature) will undergo an increase in optical power. The intraocular lens of the present invention allows the optic to undergo a change in one or both surface curvatures to undergo an increase in optical power.

Retainer plate 124 is preferably fenestrated, or is porous or perforated. Such pores 123 are depicted in FIG. 1. Fenestrated retainer plate 124 enables optical lens 122 to remain relatively undistorted when force is applied at one or more locations on retainer plate 124 as a result of accommodation pressures. In addition to its accommodation capabilities, retainer plate 124 also functions to inhibit fibrous growth by creating a mote surrounding optical lens 122. The diameter of retainer plate 124 may vary, depending upon the size and shape of the patient's eye. Typically, the outer diameter of retainer plate 124 is from about 8 mm to about 15 mm.

Optical lens 122 of intraocular lens 120 is also preferably constructed of a flexible material as described supra to allow accommodation by flexing with ciliary muscle contraction and permit changes is surface curvature. Optical lens 122 may take on a variety of shapes and sizes. For example, optical lens 122 may be spherical, aspherical, or cylindrical. The shape and size of optical lens 122 is at least partially dependent upon the optical needs of the patient. The diameter of optical lens 122 may vary, depending upon the size and shape of the patient's eye, as well as the patient's optical needs. Typically, the diameter of optical lens 122 is from about 4 mm to about 6.5 mm.

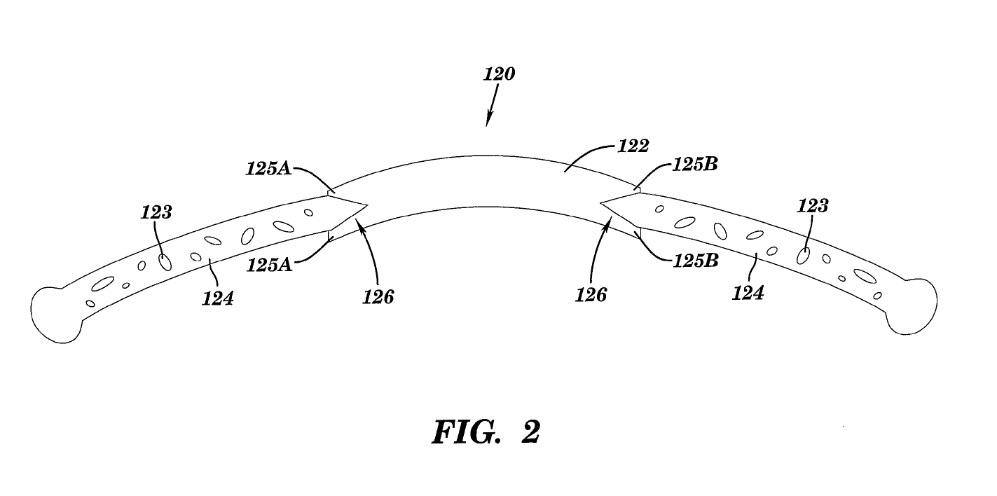

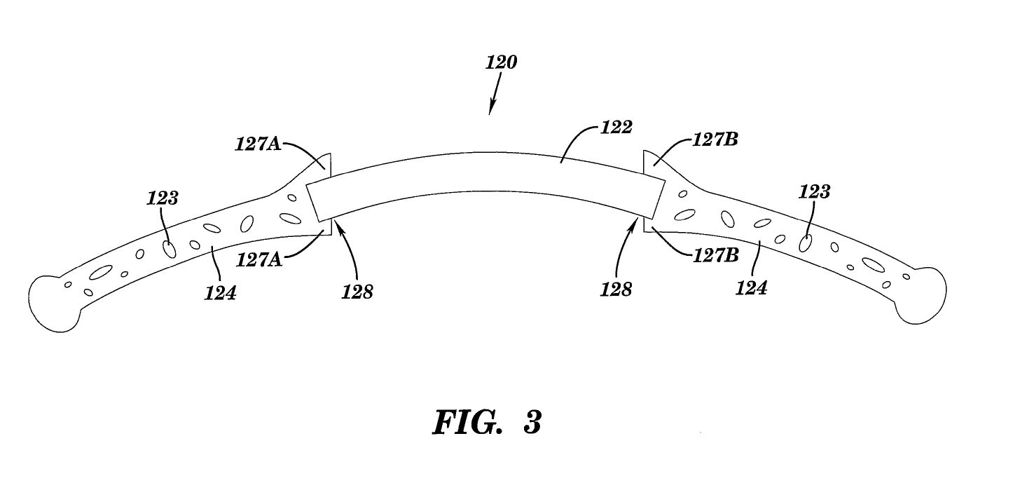

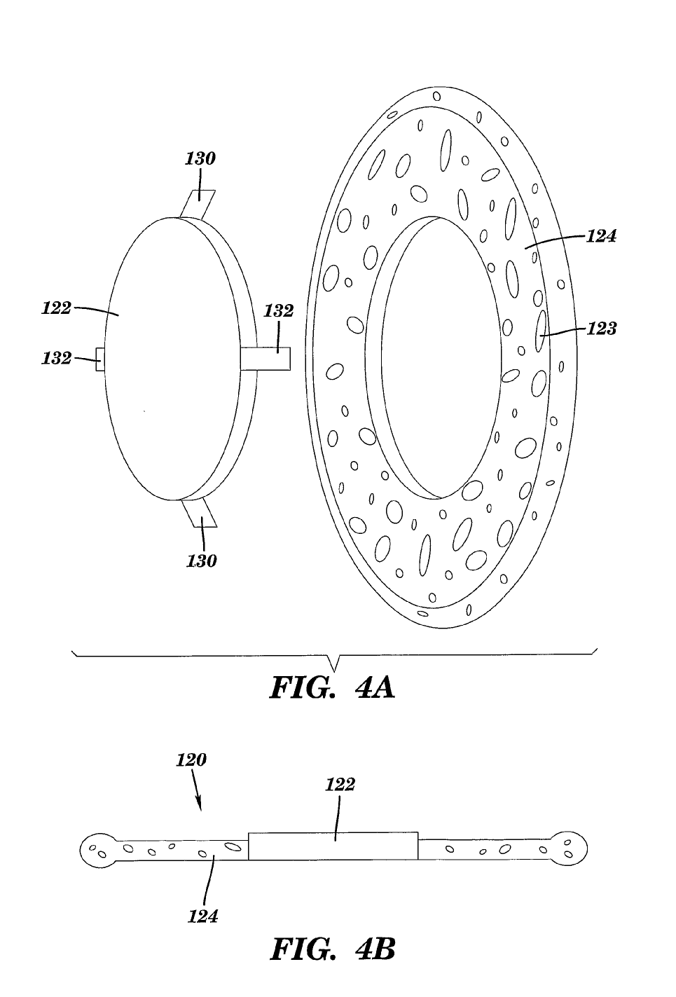

Optical lens 122 may be attached to retainer plate 124 by a variety of designs. Exemplary attachment designs include, but are not limited to, those illustrated in FIGS. 2-4. In FIG. 2, optical lens 122 has annular groove 126, defined by fingers 125A and 125B, into which retainer plate 124 fits. FIG. 3 illustrates an alternative attachment of optical lens 122 to retainer plate 124 in which retainer plate 124 has annular groove 128, defined by fingers 127A and 127B, into which optical lens 122 fits. In FIGS. 4A-B, optical lens 122 is attached to retainer plate 124 by anterior flanges 132 and posterior flanges 130. Anterior flanges 132 prevent posterior displacement of optical lens 122 and posterior flanges 130 prevent anterior displacement of optical lens 122. When such flanges are employed to attach optical lens 122 to retainer plate 124, optical lens 122 protrudes from the plane defined by retainer plate 124, as shown in FIG. 4B, to prevent fibrous growth on optical lens 122.

The intraocular lens of the present invention can be provided with a medication. Suitable medications include, without limitation, antifibroplastic agents, cyclosporine, and mitomycin.

A further aspect of the present invention relates to a method of implanting an intraocular lens into a patient's eye. This method involves providing an intraocular lens having a retainer plate with an annular region surrounding a central opening and an optical lens removably attached to the retainer plate within the central opening. The intraocular lens is implanted into the patient's eye.

The surgical procedures for implanting an accommodating intraocular lens into a patient's eye are discussed in great detail in U.S. Pat. No. 5,674,282 to Cumming, which is hereby incorporated by reference in its entirety.

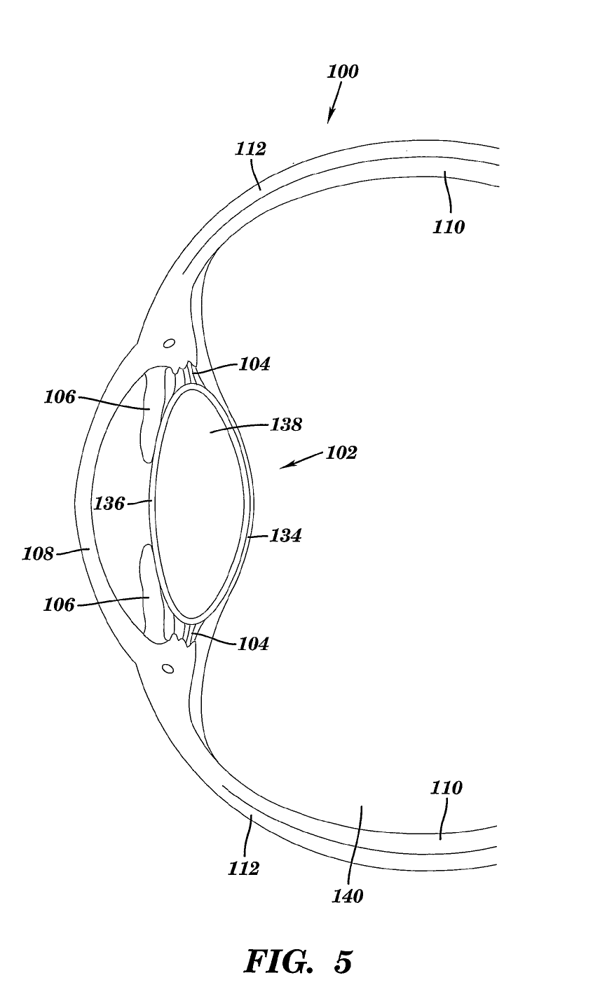

FIG. 5 illustrates a cross-sectional view of a human eye. Human eye 100 has natural crystalline lens 102. Lens 102 is contained in a capsule having anterior wall 136 and posterior wall 134, also known as anterior and posterior capsules. Anterior wall 136 and posterior wall 134 contain crystalline lens matrix 138, which in its normal state is clear, and in its cataract stage is cloudy. Prior to implanting an intraocular lens of the present invention into a patient's eye, lens matrix 138 must be removed. Surgical implantation of an intraocular lens therefore involves removing and replacing lens matrix 138 with an artificial intraocular lens.

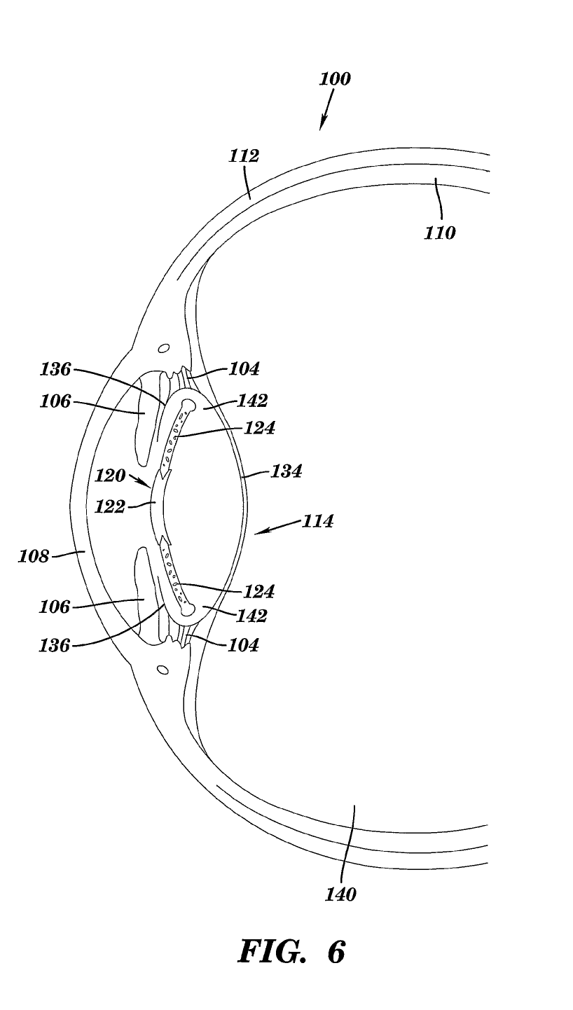

Replacement of a crystalline lens with an intraocular lens generally requires surgery in which anterior wall 136 is torn along a circular tear line, or capsulorhexis, in the center of anterior wall 136. Lens matrix 138 is removed through the capsulotomy. FIG. 6 shows a cross-section of eye 100 after lens matrix 138 has been removed. Anterior cornea 108, opaque sclera 112, retina 110, iris 106, and vitreous cavity 140 remain in eye 100. Capsular bag 114 is what is left from removal of lens matrix 138, and it remains intact within the eye. A remnant of anterior wall 136 remains after removal of lens matrix 138 as annular anterior capsular rim 136 (FIG. 6). Posterior wall 134 is joined to anterior capsular rim 136 along the perimeter of capsular bag 114 to form annular crevice-like capsular bag sulcus 142 between rim 136 and posterior wall 134. Capsular bag 114 is secured about its perimeter to the ciliary muscle of the eye by zonular ligaments 104.

FIG. 6 shows implantation of intraocular lens 120 into eye 100. Intraocular lens 120 is implanted within capsular bag 114, such that retainer plate 124 resides, at least partially, within capsular bag sulcus 142 between rim 136 and posterior wall 134. During the post-operative healing period following surgical implantation of intraocular lens 120, epithelial cells under rim 136 of capsular bag 114 cause fusion of rim 136 to posterior wall 134 by fibrosis. Fibrosis embeds retainer plate 124 within capsular bag 114, and thereby holds intraocular lens 120 in place. By implanting intraocular lens 120 into the remnants of capsular bag 114, it benefits from the accommodation forces of the eye as would a natural lens, including ciliary muscle action, zonular, and vitreous flexing. As such, intraocular lens 120 is able to make adjustments which facilitate focusing on far and near objects. Given the improved features of the intraocular lens of the present invention, including construction with flexible material and a fenestrated retainer plate, it also has the accommodation ability of surface curvature.

Implantation of the intraocular lens of the present invention may be carried out by attaching the optical lens to the retainer plate while the optical lens and the retainer plate are in the patient's eye. The retainer plate may first be inserted into the capsule and then the optical lens is inserted into the capsule, or visa versa. Preferably, the retainer plate is folded prior to insertion into the capsule. Inserting the retainer plate into the capsule may be carried out by rolling the retainer plate and inserting it into the capsular bag through a finite diameter (3-4 mm) injector. After the retainer plate and optical lens have been inserted into the eye, the optical lens may be attached to the retainer plate in the capsule of the eye.

In a preferred embodiment, a medication as described supra is applied to the intraocular lens prior to implanting the intraocular lens into the patient's eye.

Prior to implantation into a patient's eye, the intraocular lens of the present invention can be custom designed or fit for the physical dimensions and/or optical needs of the patient. Thus, retainer plate 124 is selected to fit the physical dimensions of the patient's eye by selecting a suitable diameter. Likewise, optical lens 122 is selected to fit the optical needs of the patient's eye as well as the particular retainer plate selected to fit the patient's physical dimensions.

Upon selecting the appropriate retainer plate and optical lens, optical lens 122 is attached to retainer plate 124 within the central opening of retainer plate 124 prior to implantation. Intraocular lens 120 is then implanted into the patient's eye. Depending upon the particular embodiment of the intraocular lens of the present invention, the optical lens is attached to the retainer plate by providing the optical lens with an annular groove into which the retainer plate fits, by providing the retainer plate with an annular groove into which the optical lens fits, or by securing the optical lens to the retainer plate by flanges attached to the anterior and posterior surfaces of the optical lens. See FIGS. 2-4.

After intraocular lens 120 is implanted into the patient's eye, it may later be desirable to remove optical lens 122 from retainer plate 124 and replace it with a new optical lens. For example, removal of the optical lens may have special implications for pediatric cases where the power of the optical lens can be periodically adjusted as the patient ages and the eye grows. Thus, the methods of the present invention further relate to a method of removing an optical lens from the retainer plate after the intraocular lens has been implanted into the patient's eye and attaching a replacement optical lens to the retainer plate within the central opening while the intraocular lens is in the patient's eye.

A further aspect of the present invention relates to a method of replacing an intraocular lens that has been implanted into a patient's eye. This method involves removing the intraocular lens from the patient's eye, the intraocular lens having a retainer plate with an annular region surrounding a central opening and a first optical lens removably attached to the retainer plate within the central opening. The first optical lens is removed from the retainer plate. A second optical lens is attached to the retainer plate within the central opening. The intraocular lens with the second optical lens is implanted into the patient's eye.

In carrying out this method, it is preferable to select the second optical lens to fit the optical needs of the patient's eye prior to attaching the second optical lens to the retainer plate. Furthermore, it is desirable to apply a medication as described supra to the second optical lens prior to implanting the intraocular lens with the second optical lens into the patient's eye.

The patient may be any mammal, such as, without limitation, horses, dogs, cats, rabbits, and small rodents. Other mammals such as pigs, sheep, cows, and other livestock may also be patients for purposes of the present invention. In a preferred embodiment, the patient is human.

Another aspect of the present invention relates to an intraocular lens having a retainer plate with an annular region surrounding a central opening, wherein the annular region is porous or perforated, and an optical lens integral with the retainer plate within the central opening.

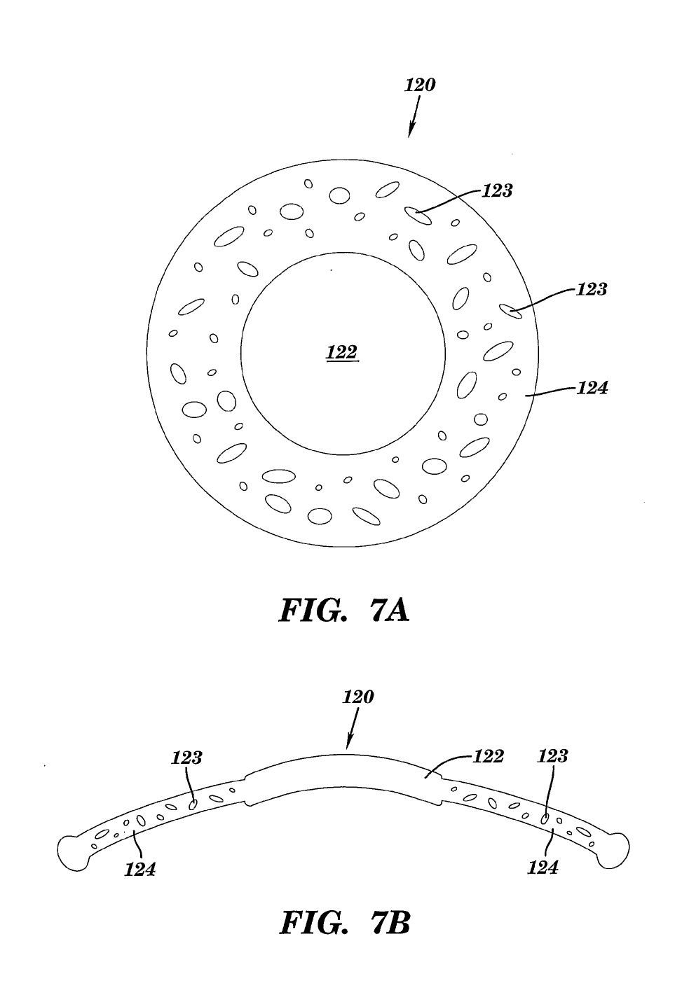

FIGS. 7A and 7B depict intraocular lens 120 having retainer plate 124, which has pores 123. Optical lens 122 is integral with retainer plate 124. Preferably, retainer plate 124 and optical lens 122 are made from a single piece of material. Suitable materials for construction of intraocular lens 120 are discussed supra.

Yet another aspect of the present invention relates to a method of implanting an intraocular lens into a patient's eye. This method involves providing an intraocular lens having a retainer plate with an annular region surrounding a central opening, wherein the annular region is porous or perforated, and an optical lens integral with the retainer plate within the central opening. The intraocular lens is implanted into the patient's eye.

The same procedures described supra apply to carrying out this method of the present invention.

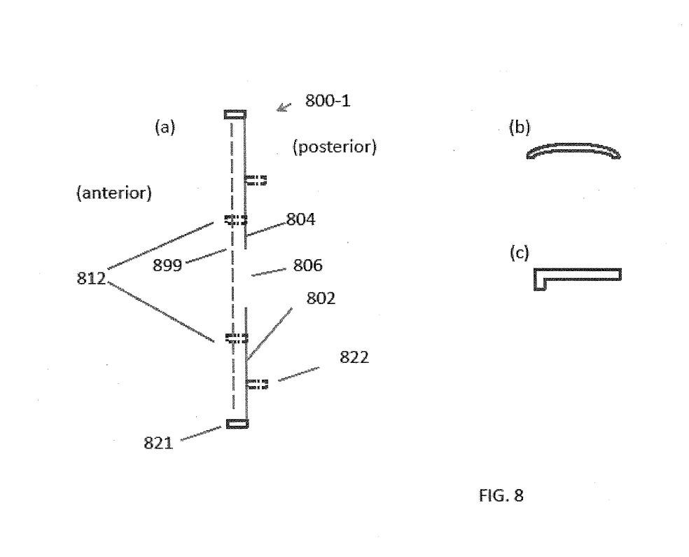

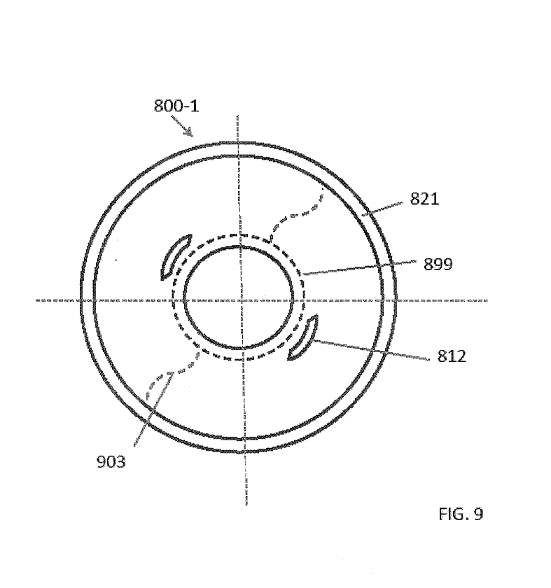

An enhancer 800-1 for an in-vivo intra-ocular lens (IOL) 899 (shown in dotted line since it is not a part of the enhancer per se), is illustrated in FIGS. 8a and 9. The enhancer includes a retainer plate 802 having an annular region 804 comprising an anterior surface and a posterior surface surrounding a central clear aperture 806. The retainer plate has a lip 821 projecting from a region of the anterior surface of the retainer plate, wherein the lip provides a contact surface for at least a portion (i.e., a lens perimetal surface or a haptic (903) of the lens) of an IOL (899). The lip 821 is continuous and is located adjacent the outer perimeter region of the retainer plate, and is centered about the clear aperture 806. The continuous lip 821 alternatively may be positioned in-between the outer and inner perimeter of the retainer plate.

The enhancer may also include at least two opposing lip segments 812, which project from the anterior surface of the retainer plate and are centered about the clear aperture 806. The lip segments 812 are located at least about 1 millimeter (mm) from the inner perimeter of the retainer plate and have an opposing spacing that will allow about 0.5 mm of play between the circumference of an IOL and the segment. These lip segments then act as nubs to keep the IOL centered in the clear aperture of the retainer plate. The circumferential spacing between the segments provides space for the lens haptics 903 to protrude out and engage the inner surface of the lip 821.

The lips 812, 821 have a thickness that is greater than the thickness of the retainer plate. For example, the lips have a thickness that is about 0.5× to about 5× of the retainer plate's thickness. The retainer has a thickness between about 50 to 100 micrometers (μm) and thus the lip has a thickness between about 100 to 500 μm.

As shown in FIG. 8a, the lips 812, 821 have flat inner contact surfaces. Alternatively, as shown in FIGS. 8b, 8c, the lips may have a curved or a ‘sideways L’ shape, or other shapes that appropriately constrain an IOL or haptic structure thereof. The shapes are designed to accommodate the wide variety of IOLs that may be used with the enhancer of the invention.

As with the retainer plate 124 described herein above, the retainer plate 802 may be perforated or porous.

The enhancer may also include a continuous or segmented lip 822 projecting from a region of the posterior surface of the retainer plate. This lip (or lip segment(s)) 822 functions to inhibit fibrous growth around the IOL.

As with the retainer plate 124 described herein above, the retainer plate 802 is characterized by a flexibility sufficient to allow its insertion into a capsular bag.

In an exemplary aspect, the retainer plate 802 has a diameter of between about 10 mm to about 15 mm. The annular region has a diameter of between about 4 mm to about 6 mm.

As with the retainer plate 124 described herein above, the retainer plate 802 is medicated. The retainer plate is medicated with at least one of an antifibroplastic agent, cyclosporine, and mitomycin.

Similarly to the retainer plate 124 shown in FIG. 3, an enhancer for an in-vivo intra-ocular lens (IOL) can include a retainer plate having an annular region comprising an anterior surface and a posterior surface surrounding a central clear aperture, wherein the retainer plate has a groove in at least a portion of the inner perimetal surface of the annular region into which a corresponding peripheral region of an IOL can engage.

A related embodiment of the invention is a method. According to an aspect, the method includes the steps of inserting an enhancer 800-1 into a capsular bag of an in-vivo, aphakic eye, and positioning an IOL (899) adjacent the anterior surface of the retainer plate in a manner such that the IOL is held in operable contact by at least a portion of the lip of the retainer plate. The IOL is advantageously an accommodating IOL. The IOL may be positioned after inserting the enhancer in the capsular bag.

All references, including publications, patent applications, and patents cited herein are hereby incorporated by reference in their entireties to the extent allowable and to the same extent as if each reference was individually and specifically indicated to be incorporated by reference and was set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

The recitation of ranges of values herein is merely intended to serve as a shorthand method of referring individually to each separate value falling within the range, unless otherwise indicated herein, and each separate value is incorporated into the specification as if it was individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not impose a limitation on the scope of the invention unless otherwise claimed.

No language in the specification should be construed as indicating any non-claimed element as essential to the practice of the invention.

It will be apparent to those skilled in the art that various modifications and variations can be made to the embodiments without departing from the spirit and scope of the invention. There is no intention to limit the invention to the specific form or forms disclosed, but on the contrary, the intention is to cover all modifications, alternative constructions, and equivalents falling within the spirit and scope of the invention, as defined in the appended claims. Thus, it is intended that the present invention cover the modifications and variations of this invention provided they come within the scope of the appended claims and their equivalents.

What is claimed:

1. An enhancer for an in-vivo intra-ocular lens (IOL), comprising:
a retainer plate having an annular region comprising an anterior surface and a posterior surface surrounding a central clear aperture;
a lip projecting from a region of the anterior surface of the retainer plate, wherein the lip provides a contact surface for at least a portion of an IOL.

2. The enhancer of claim 1, wherein the lip is located adjacent an outer perimeter region of the retainer plate.

3. The enhancer of claim 1, wherein the lip is continuous and centered about the clear aperture.

4. The enhancer of claim 1, wherein the lip is segmented.

5. The retainer of claim 1, wherein the lip is located adjacent an inner perimeter of the retainer plate.

6. The enhancer of claim 5, wherein the lip is located at least about 1 millimeter (mm) from the inner perimeter of the retainer plate.

8. The enhancer of claim 1, wherein the lip is located intermediate the outer perimeter and the inner perimeter of the retainer plate.

9. The enhancer of claim 4, wherein the lip comprises at least two segments disposed in opposing relationship and centered about the clear aperture.

10. The enhancer of claim 9, wherein the at least two opposing lip segments are located at least about 1 millimeter (mm) from the inner perimeter of the retainer plate.

11. The enhancer of claim 1, further comprising a lip projecting from a region of the posterior surface of the retainer plate.

12. The enhancer of claim 11, further comprising at least one lip segment.

13. The enhancer of claim 1, wherein the retainer plate is characterized by a flexibility sufficient to allows its insertion into a capsular bag.

14. The enhancer of claim 1, wherein the lip has a thickness that is greater than the thickness of the retainer plate.

15. The enhancer of claim 1, wherein the lip has a thickness that is about 0.5× to about 5× of the retainer plate's thickness.

16. The enhancer of claim 14, wherein the retainer has a thickness between about 50 to 100 micrometers (μm) and the lip has a thickness between about 100 to 500 μm.

17. The enhancer of claim 1, wherein the lip has a flat inner surface.

18. The enhancer of claim 1, wherein the lip has a hooked shape.

19. The enhancer of claim 1, wherein the retainer plate is perforated.

20. The enhancer of claim 1, wherein the retainer plate has a diameter of between about 10 mm to about 15 mm.

21. The enhancer of claim 1, wherein the annular region has a diameter of between about 4 mm to about 6 mm.

22. The enhancer of claim 1, wherein the retainer plate is medicated.

23. The enhancer of claim 22, wherein the retainer plate is medicated with at least one of an antifibroplastic agent, cyclosporine, and mitomycin.

24. An enhancer for an in-vivo intra-ocular lens (IOL), comprising:
a retainer plate having an annular region comprising an anterior surface and a posterior surface surrounding a central clear aperture, wherein the retainer plate has a groove in at least a portion of the inner perimetal surface of the annular region into which a corresponding peripheral region of an IOL can engage.

25. A method, comprising:
inserting an enhancer according to claim 1 into a capsular bag of an in-vivo, aphakic eye; and
positioning an IOL adjacent the anterior surface of the retainer plate in a manner such that the IOL is held in operable contact by at least a portion of the lip of the retainer plate.

26. The method of claim 26, wherein the IOL is an accommodating IOL.

27. The method of claim 26, further comprising positioning the IOL after inserting the enhancer.
